(19) United States
(12) Patent Application Publication
(10) Pub. No.: US 2010/0076101 A1
(43) Pub. Date: Mar. 25, 2010

(54) POLYPHENYLENEPOLYMETHYLENE POLYISOCYANATE AND ITS USE FOR PRODUCING POLYURETHANE FOAMS

(75) Inventors: Hans-Juergen Reese, Damme (DE); Imbridt Murrar, Senftenberg (DE); Ralf Fritz, Bissendorf-Schledehausen (DE); Andres Cabrera, Osnabrueck (DE); Birgit Magg, Damme (DE); Baerbel Guschel, Lauchhammer-Nord (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, Ludwigshafen (DE)

(21) Appl. No.: 12/513,515

(22) PCT Filed: Nov. 12, 2007
(86) PCT No.: PCT/EP07/62188

§ 371 Date: May 5, 2009

(30) Foreign Application Priority Data
Nov. 17, 2006 (EP) 06124306.9

(51) Int. Cl. C08G 18/06; C07C 265/00

(52) U.S. Cl. 521/128; 560/355

(57) ABSTRACT

The invention relates to polyphenylenepolymethylene polyisocyanates (B) comprising
(B1) the 2-ring product of polyphenylenepolymethylene polyisocyanate
(B2) the 3-ring product of polyphenylenepolymethylene polyisocyanate
(B3) the 4-ring product of polyphenylenepolymethylene polyisocyanate
(B4) the 5-ring product of polyphenylenepolymethylene polyisocyanate,
wherein the constituents (B2), (B3) and (B4) are, at a content of (B1) of up to 55% by weight, based on the weight of (B), present in a weight ratio of (B2):(B3):(B4) of 8±4:3.5±1.8:1.2±0.9 and the component (B) comprises at least 85% by weight, based on the weight of the component (B), of the constituents (B1), (B2), (B3) and (B4).

The invention provides a polyphenylenepolymethylene polyisocyanate (MDI) having a particular composition, a process for preparing it and its uses for producing polyurethanes, in particular polyurethane foams.

Polyurethane foams have been known for a long time and have been described many times. They can be used for many industrial applications. They are usually produced by reacting polyisocyanates with compounds having at least two hydrogen atoms which are reactive toward isocyanate groups.

Two classes of frequently used polyurethane foams are rigid polyurethane foams and 1-component foams, also referred to as aerosol foams.

Rigid polyurethane foams are used predominantly for thermal insulation, for example in refrigeration appliances, transport means or buildings and also for producing structural elements, in particular sandwich elements.

Polyisocyanates used in the production of the polyurethanes mentioned are usually aromatic polyisocyanates, in particular MDI and its higher homologues.

One-component foams from aerosol containers are installation materials which are frequently used in building and construction for installing windows and doors in buildings and also as filling material for hollow spaces caused by the method of construction or holes through walls for pipe installations. Such an aerosol container comprises a prepolymer and also blowing agents and additives. The desired foam is formed by discharge of the contents of the container by means of the blowing agents, foaming by frothing and curing by contact with atmospheric moisture. One-component foams based on NCO-comprising prepolymers are the best known foams of this type. There are here a variety of products which, depending on the composition, lead to rigid to flexible foams.

An important requirement which the polyurethane foams have to meet is dimensional stability. Dimensional stability means that the foam does not change its volume after curing, in particular does not shrink. In the case of rigid foams, shrinkage can result in voids in the foam and detachment from the covering layers.

In the case of one-component foams for installation of windows and doors, shrinkage can lead to unsatisfactory stability of the doors and windows which have been installed.

The problem of dimensional stability, in particular of one-component foams, has not been fully solved industrially, so that, for the grades known up to now, a shrinkage of up to 5% in room-temperature applications and a shrinkage of up to 10% at 40° C. and 90% relative humidity in tropical applications are still permissible as technically unavoidable shrinkage values.

In the case of two-component foams, particularly in the case of two-component rigid foams, the shrinkage problem exists particularly in the case of large moldings, and there are no pointers to a solution to this.

Furthermore, the market is increasingly demanding foams which have a light color. The foams offered up to now, which have been produced using the customarily used polyphenylenepolymethylene polyisocyanates, usually have a brown color. This can be considered to be unsatisfactory, particularly in applications in which the foam is visible.

It was therefore an object of the invention to provide polyurethane foams which have good processing properties and use properties, in particular a good dimensional stability. Furthermore, market demands for light-colored foams should be addressed. The process should allow foams for various applications, in particular one-component in-situ foams and rigid polyurethane foams, to be produced.

This object has surprisingly been able to be achieved by the use of a mixture of diphenylmethane diisocyanates and polyphenylenepolymethylene polyisocyanates having a specific composition as isocyanate components in the production of the foams.

The invention accordingly provides a polyphenylenepolymethylene polyisocyanate (B) comprising

(B1) the 2-ring product of polyphenylenepolymethylene polyisocyanate

(B2) the 3-ring product of polyphenylenepolymethylene polyisocyanate

(B3) the 4-ring product of polyphenylenepolymethylene polyisocyanate

(B4) the 5-ring product of polyphenylenepolymethylene polyisocyanate,

wherein the constituents (B2), (B3) and (B4) are, at a content of (B1) of up to 55% by weight, based on the weight of (B), present in a weight ratio of (B2):(B3):(B4) of 8±4:3.5±1.8:1.2±0.9 and the component (B) comprises at least 85% by weight, based on the weight of the component (B), of the constituents (B1), (B2), (B3) and (B4).

The invention further provides a process for producing polyurethane foams by reacting (A) compounds having at least two hydrogen atoms which are reactive toward isocyanate groups, hereinafter also referred to as polyol components, with (B) polyisocyanates, wherein the polyphenylenepolymethylene polyisocyanate of the invention is used as polyisocyanate (B).

The invention further provides a process for preparing the polyphenylenepolymethylene polyisocyanate of the invention, which comprises the steps
a) preparation of polyphenylenepolymethylene polyisocyanate by reacting polyphenylenepolymethylenepolyamine with phosgene,
b) removal of by-products from the polyphenylenepolymethylene polyisocyanate from step a).

The polyphenylenepolymethylene polyisocyanate of the invention has further constituents in addition to the components (B1) to (B4). Thus, the polyphenylene-polymethylene polyisocyanate of the invention (B) further comprises polyphenylenepolymethylene polyisocyanate having 6 or more rings. For the present purposes, the term “ring” refers to an aromatic ring. The compounds comprising more than two aromatic rings can in the following also be referred to as higher homologues.

The polyphenylenepolymethylene polyisocyanate (B) of the invention can additionally comprise other compounds comprising isocyanate groups, e.g. reaction products of isocyanates with one another, in particular uretonimines, and/or polyphenylene-polymethylene polyisocyanates having 6 or more rings.

The proportion of such further constituents of the component (B) is preferably not more than 15% by weight, based on the weight of the component (B).

The polyphenylenepolymethylene polyisocyanate (B) preferably comprises not more than 11% by weight, particularly preferably not more than 6% by weight and in particular not more than 3% by weight, in each case based on the weight of (B), of uretonimines. These are part of the 15% by weight, based on the weight of (B), of the other compounds.

The determination of the contents of polyphenylenepolymethylene polyisocyanates having different ring contents is usually carried out by means of gas chromatography. The content of uretonimines in the polyphenylenepolymethylene polyisocyanate is determined by means of FT-IR analysis on the basis of a calibration with 3-ring uretonimine (test method PFO/A 00/22-03).

The polyphenylenepolymethylene polyisocyanate of the invention preferably has a content of free NCO end groups of from 31.0 to 33.3% by weight.

The polyphenylenepolymethylene polyisocyanate (B) of the invention which has been obtained by means of extraction preferably has an iodine color number of less than 5 iodine, an L* value of greater than 96 and a b* value of less than 15, determined in accordance with DIN 6162 and DIN 6164.

The polyphenylenepolymethylene polyisocyanates of the invention can be prepared by conventional methods. These are generally known and comprise the preparation of diphenylmethanediamine (MDA) and its higher homologues by acid-catalyzed reaction of aniline and formaldehyde, neutralization and work-up of the amine mixture obtained in this way, reaction of the latter with phosgene to form polyphenylenepolymethylene polyisocyanate and purification, work-up and, if appropriate, partial removal of the 2-ring MDI. It has surprisingly been found that damage to the polyphenylenepolymethylene polyisocyanate due to formation of by-products occurs, in particular, as a result of the thermal stress in the work-up of the polyphenylene-polymethylene polyisocyanate and the removal of the 2-ring products by distillation. These disadvantages can be avoided if the thermal stress occurs for a very brief period, for example when a smaller proportion of the 2-ring MDI is separated off. Furthermore, it is important that the temperature in the process is not increased to values above 220° C. The polyphenylenepolymethylene polyisocyanate according to the invention (B) prepared in this way preferably has an iodine color number of less than 10 iodine, an L* value of greater than 89 and a b* value of less than 30, determined in accordance with DIN 6162 and DIN 6164.

The polyphenylenepolymethylene polyisocyanates of the invention are prepared in a preferred process by firstly reacting polyphenylenepolymethylenepolyamine with phosgene in a customary and known way in a process step a) and freeing the product of by-products, for example uretonimines, in a subsequent process step b). Alternative process routes are also possible in principle if they lead to the same products.

The process step a) is generally known and comprises, as described above, the acid-catalyzed reaction of aniline with formaldehyde, neutralization and work-up of the polyamine formed, reaction of the latter with phosgene to form the corresponding polyisocyanate and work-up and purification of the latter.

As described, the polyphenylenepolymethylene polyisocyanate of the invention is freed of secondary compounds such as uretonimine in step b). These secondary compounds are formed in the preparation and work-up, in particular by thermal stressing of the polyisocyanate. These secondary compounds from the production process, e.g. uretdiones, uretonimines, carbamoyl chlorides, are comprised in the starting polyisocyanate in a maximum amount of 25% by weight. They are preferably removed by liquid-liquid extraction with polar or nonpolar solvents. In a preferred embodiment, hydrocarbons such as cyclohexane are preferred as solvents. Such processes are described, for example, in DE 1,543,258 or EP 133 538.

In a preferred embodiment of step b), the polyphenylenepolymethylene polyisocyanate used is brought into contact with cyclohexane in a ratio of isocyanate:solvent of from 1:1 to 1:15, preferably from 1:1.5 to 1:12 and particularly preferably from 1:2.5 to 1:10 at a temperature of from 20 to 90° C., preferably from 30 to 80° C., for from 1 to 180 minutes, preferably from 5 to 150 minutes. The product mixture is then allowed to stand at from 20 to 40° C., preferably at room temperature, until phase separation is complete. The lower phase is the “raffinate” which comprises the uretonimine to be separated off and also higher-ring MDI homologues. The upper phase is the “extract” which comprises the desired low-uretonimine polyphenylenepolymethylene polyisocyanate and solvent. The two phases are separated and the solvent is removed virtually completely, for example by means of vacuum distillation. The residual cyclohexane content is preferably less than 20 ppm.

The polyphenylenepolymethylene polyisocyanate according to the invention (B) obtained by means of extraction preferably has an iodine color number of less than 5 iodine, an L* value of greater than 96 and a b* value of less than 15, determined in accordance with DIN 6162 and DIN 6164.

Particularly good results are achieved when the thermal stress on the polyphenylene-polymethylene polyisocyanate is kept low in the preparation and an extraction of the polyphenylenepolymethylene polyisocyanate is additionally carried out.

It is also possible to subject polyphenylenepolymethylene polyisocyanate batches which do not have the composition according to the invention to an extraction. Here, the content of secondary compounds can be reduced from above 15% by weight to the content according to the invention. In addition, it is possible to shift the ring distribution in the direction of low-ring products in this way. The extraction of the polyphenylenepolymethylene polyisocyanate can also be carried out subsequent to the partial removal of 2-ring MDI, since an increase in the content of secondary compounds frequently takes place here.

The polyphenylenepolymethylene polyisocyanate according to the invention (B) which has been obtained by means of the above-described process features in the preparation of polyphenylenepolymethylene polyisocyanate preferably has a content of 2-ring product (B1) of from 20 to 50% by weight, based on the weight of (B), with the constituents (B2), (B3) and (B4) being present in a weight ratio of (B2):(B3):(B4) of 8±4:3.5±1.8:1.2±0.9 and the component (B) comprising at least 85% by weight, based on the weight of the component (B), of the constituents (B1), (B2), (B3) and (B4).

When the polyphenylenepolymethylene polyisocyanate according to the invention (B) has been obtained by extraction, it preferably has a content of 2-ring product (B1) of from 20 to 55% by weight, based on the weight of (B), with the constituents (B2), (B3) and (B4) being present in a weight ratio of (B2):(B3):(B4) of 8±4:3.5±1.8:1.2±0.9 and the component (B) comprising at least 85% by weight, based on the weight of the component (B), of the constituents (B1), (B2), (B3) and (B4).

When the polyphenylenepolymethylene polyisocyanate according to the invention (B) has been obtained by partial removal of the 2-ring MDI and subsequent extraction, it preferably has a content of 2-ring product (B1) of from 2 to 20% by weight, based on the weight of (B), with the constituents (B2), (B3) and (B4) being present in a weight ratio of (B2):(B3):(B4) of 8±4:3.5±1.8:1.2±0.9 and the component (B) comprising at least 85% by weight, based on the weight of the component (B), of the constituents (B1), (B2), (B3) and (B4).

The polyphenylenepolymethylene polyisocyanate of the invention can, as described, be used for producing polyurethane foams. Preferred applications here are 1-component polyurethane foams and rigid polyurethane foams. For this purpose, the polyphenylenepolymethylene polyisocyanate of the invention (B) is reacted with compounds having at least two hydrogen atoms which are reactive toward isocyanate groups (A).

In the production of 1-component polyurethane foams, the reaction of the isocyanate component (B) with the compounds having at least two hydrogen atoms which are reactive toward isocyanate groups (A) takes place in the presence of a blowing agent in a pressure container, preferably an aerosol can. For this purpose, the polyol component (A) and the isocyanate component (B) are introduced in the ratio indicated above together with a blowing agent into a pressure container, so that the prepolymer according to the invention having a lower content of free isocyanate groups is formed in the pressure container. Customary blowing agents for producing 1-component in-situ foams are, for example, R134a (tetrafluoroethane), R152a (1,1-difluoroethane), dimethyl ether, propane, n-butane, isobutane, preferably mixtures of propane, n-butane and isobutane.

The NCO content of the prepolymers present in the aerosol can is preferably in the range from about 5 to 28% by weight, preferably from 8 to 24% by weight, particularly preferably from 9 to 18% by weight. Prepolymers having a lower NCO content lead to more flexible aerosol foams while those having a higher NCO content correspondingly lead to more rigid foams.

To apply the 1-component polyurethane foams, the pressure container is depressurized. Here, the exiting prepolymer is foamed by the frothing action of the blowing agent and cures by contact with atmospheric moisture.

For many applications of the 1-component polyurethane foams, it is necessary to add flame retardants as additives and these likewise have the effect of lowering the viscosity. They have to be added only in the amount necessary to achieve the burning class of the finished foam.

Trialkyl phosphates and trichloroalkyl phosphates are usually used as added flame retardants. The alkyl radicals preferably have from 1 to 4, particularly preferably from 1 to 3, carbon atoms. Particularly preferred compounds are trimethyl phosphate, triethyl phosphate, tripropyl phosphate, trichloromethyl phosphate, trichloroethyl phosphate and trichloropropyl phosphate. These can be used individually or in any mixtures with one another.

The amount of flame retardants added depends on the requirements which the foam has to meet. For the formulation of flame-resistant 1-component polyurethane foams, the NCO prepolymer present in the can has to have a total content of added flame retardants of from about 8 to 18% by weight, preferably from 12 to 16% by weight, based on the weight of the prepolymer. At lower contents, the flame protection can be unsatisfactory, while at high contents of added flame retardants, the foam flows, i.e. it cannot be applied to a vertical surface, or a large proportion of the flame retardant migrates out of the foam.

In the production of the rigid foams according to the invention, an isocyanate component, in which the polyphenylenepolymethylene polyisocyanate of the invention (B) is used in the present process, is, as generally known, reacted with the compounds having at least two hydrogen atoms which are reactive toward isocyanate groups (A) in the presence of catalysts and blowing agents.

As blowing agent, it is possible to use water which reacts with isocyanate groups to eliminate carbon dioxide. In combination with or in place of water, it is also possible to use physical blowing agents. These are compounds which are inert toward the starting components and are usually liquid at room temperature and vaporize under the conditions of the urethane reaction. The boiling point of these compounds is preferably below 50° C. Physical blowing agents also include compounds which are gaseous at room temperature and are introduced under pressure into the starting components or are dissolved therein, for example carbon dioxide, low-boiling alkanes and fluoroalkanes.

The compounds are usually selected from the group consisting of alkanes and/or cycloalkanes having at least 4 carbon atoms, dialkyl ethers, esters, ketones, acetals, fluoroalkanes having from 1 to 8 carbon atoms and tetraalkylsilanes having from 1 to 3 carbon atoms in the alkyl chain, in particular tetramethylsilane.

Examples which may be mentioned are propane, n-butane, isobutane and cyclobutane, n-pentane, isopentane and cyclopentane, cyclohexane, dimethyl ether, methyl ethyl ether, methyl butyl ether, methyl formate, acetone and also fluoroalkanes which are degraded in the troposphere and therefore do not harm the ozone layer, e.g. trifluoromethane, difluoromethane, 1,1,1,3,3-pentafluorobutane, 1,1,1,3,3-pentafluoro-propane, 1,1,1,2-tetrafluoroethane, difluoroethane and heptafluoropropane. Particular preference is given to using cyclopentane and/or n-pentane. The physical blowing agents mentioned can be used either alone or in any combinations with one another.

To produce the rigid polyurethane foams, the polyol component (A) and the polyisocyanates (B) are reacted in such amounts that the isocyanate index is in the range from 100 to 220, preferably from 125 to 195.

The rigid polyurethane foams can be produced batchwise or continuously with the aid of known mixing apparatuses.

The rigid PUR foams according to the invention are usually produced by the two-component process. In this process, the compounds having at least two hydrogen atoms which are reactive toward isocyanate groups (A) are mixed with the flame retardants, the blowing agents, the catalysts and the further auxiliaries and/or additives to form the polyol component and this is reacted with the polyisocyanates or mixtures of the polyisocyanates and, if appropriate, flame retardants and blowing agents, also referred to as isocyanate component.

The starting components are usually mixed at a temperature of from 15 to 35° C., preferably from 20 to 30° C. The reaction mixture can be introduced into closed support tools by means of high-or low-pressure metering machines. Sandwich elements are manufactured, e.g. batchwise, by means of this technology.

The foams produced by the process of the invention surprisingly have a very light, sometimes even white, color. The foams are dimensionally stable and can be applied very well.

As regards the other polyols for producing the polyurethane foams according to the invention, the following details may be provided:

Compounds having at least two hydrogen atoms which are reactive toward isocyanate (A) which can be used in the process of the invention for the production of rigid foams and for the preparation of the prepolymers for 1-component in-situ foams are, in particular, polyether alcohols and/or polyester alcohols having OH numbers in the range from 100 to 1200 mg KOH/g.

The polyester alcohols used are usually prepared by condensation of polyfunctional alcohols, preferably diols, having from 2 to 12 carbon atoms, preferably from 2 to 6 carbon atoms, with polyfunctional carboxylic acids having from 2 to 12 carbon atoms, for example succinic acid, glutaric acid, adipic acid, suberic acid, azelaic acid, sebacic acid, decanedicarboxylic acid, maleic acid, fumaric acid and preferably phthalic acid, isophthalic acid, terephthalic acid and the isomeric naphthalenedicarboxylic acids.

The polyether alcohols used usually have a functionality of from 2 to 8, in particular from 3 to 8.

In particular, polyether polyols prepared by known methods, for example by cationic polymerization of alkylene oxides in the presence of catalysts, preferably alkali metal hydroxides, are used.

As alkylene oxides, use is usually made of ethylene oxide and/or propylene oxide, preferably pure 1,2-propylene oxide.

Starter molecules used are, in particular, compounds having at least 3, preferably from 4 to 8, hydroxyl groups or at least two primary amino groups in the molecule.

As starter molecules having at least 3, preferably from 4 to 8, hydroxyl groups in the molecule, preference is given to using trimethylolpropane, glycerol, pentaerythritol, sugar compounds such as glucose, sorbitol, mannitol and sucrose, polyhydric phenols, resols such as oligomeric condensation products of phenol and formaldehyde and Mannich condensates of phenols, formaldehyde and dialkanolamines and also melamine.

As starter molecules having at least two primary amino groups in the molecule, preference is given to using aromatic diamines and/or polyamines, for example phenylenediamines, 2,3-, 2,4-, 3,4- and 2,6-toluenediamine and 4,4′-, 2,4′- and 2,2′-diaminodiphenylmethane and also aliphatic diamines and polyamines, e.g. ethylenediamine.

The polyether polyols have a functionality of preferably from 3 to 8 and hydroxyl numbers of preferably from 100 mg KOH/g to 1200 mg KOH/g and in particular from 240 mg KOH/g to 570 mg KOH/g. It is also possible for polyols, in particular polyether alcohols, having a hydroxyl number of less than 100 mg KOH/g and a functionality of from 2 to 3 to be additionally used. In this way, the properties of the foams can be adjusted, for example in the case of 1-component in-situ foams in the direction of higher flexibility.

Compounds having at least two hydrogen atoms which are reactive toward isocyanate (A) also include the chain extenders and crosslinkers which may be concomitantly used. The addition of bifunctional chain extenders, trifunctional and higher-functional crosslinkers or, if appropriate, mixtures thereof can prove to be advantageous for modifying the mechanical properties. As chain extenders and/or crosslinkers, preference is given to using alkanolamines and in particular diols and/or triols having molecular weights of less than 400, preferably from 60 to 300.

Chain extenders, crosslinkers or mixtures thereof are advantageously used in an amount of from 1 to 20% by weight, preferably from 2 to 5% by weight, based on the polyol component (A).

Apart from the polyester and polyether polyols, chain extenders and crosslinkers mentioned, monools can be used as further OH-functional compounds as targeted agent for regulating the molecular weight in the manufacture of the polyol component (A) required in the field of 1-component polyurethane foams in order to form a permeable foam skin and ultimately to improve the storage stability. These monools having molecular weights up to 1400 g/mol (OH number: about 40 mg KOH/g) are usually, if required, used in proportions of up to 10% by weight, based on the polyol component (A).

Further information on the polyether alcohols and polyester alcohols used and their preparation may be found, for example, in Kunststoffhandbuch, volume 7 “Polyurethane”, edited by Günter Oertel, Carl-Hanser-Verlag, Munich, 3rd edition, 1993.

In a particularly preferred embodiment of the production of 1-component polyurethane foams, a mixture of

(A1) a polyester polyol having a molecular weight of not more than 600 g/mol and

(A2) a polyether polyol or polyether polyol mixture having a mean molecular weight of from 1000 to 5000 g/mol

is used as polyol component (A).

As polyester polyol (A1), preference is given to using a polyester polyol based on phthalic anhydride/diethylene glycol/polyethylene glycol.

The polyols (A1) and (A2) are preferably used in a weight ratio of polyester polyol (A1) to polyether polyol or polyether polyol mixture (A2) in the range from 1:6 to 3:1.

Catalysts used are, in particular, compounds which strongly accelerate the reaction of the isocyanate groups with the groups which are reactive toward isocyanate groups. Such catalysts are strongly basic amines such as secondary aliphatic amines, imidazoles, amidines and also alkanolamines.

If isocyanurate groups are to be incorporated into the rigid foam, specific catalysts are required. As isocyanurate catalysts, use is usually made of metal carboxylates, in particular potassium acetate and its solutions. In the production of such foams, also referred to as polyurethane-polyisocyanurate foams, the reaction of the components (A) and (B) is usually carried out at an index of from 160 to 450.

The invention is illustrated by the following examples.

### EXAMPLE 1

### One-Component Foam

### 1.1 Preparation of the Polyol Component:

A polyol component was prepared by mixing 300 g of a polyester polyol based on phthalic anhydride/diethylene glycol/polyethylene glycol and having a molecular weight of from 470 g/mol, 208 g of a polyether polyol based on glycerol/propylene oxide/ethylene oxide and having a molecular weight of 4000 g/mol, 30 g of a polyether polyol based on sucrose/pentanediol/diethylene glycol and having a mean molecular weight of 540 g/mol, 40 g of a polyethylene glycol having a molecular weight of 600 g/mol, 59 g of a monofunctional methylated polyethylene glycol having a molecular weight of 500 g/mol, 25 g of a foam stabilizer, 330 g of trichloropropyl phosphate, 8 g of bis(morpholinoethyl) ether, 0.5 g of silicone oil.

### 1.2 Preparation of the Isocyanate Preparations:

Polyphenylenepolymethylene polyisocyanate (trade name: Lupranat® M20) having a monomeric MDI content of 37%, an NCO content of 31.2% by weight, a viscosity of 213 mPa·s at 25° C., a color number of 20 iodine, an L* value of 85.6, a b* value of 70.1 and a uretonimine content of 8.4% by weight was extracted with cyclohexane in a single-stage extraction process as described below.

Polyphenylenepolymethylene polyisocyanate was brought into contact with cyclohexane in a ratio of isocyanate:solvent of 1:3 at 50° C. for 60 minutes. The product mixture was then allowed to stand at room temperature until phase formation was complete.

The upper phase was the “extract” which comprised the desired polyphenylene-polymethylene polyisocyanate and solvent. The solvent was removed completely from the extract by means of vacuum distillation (residual cyclohexane content less than 20 ppm).

This gave a product which can be characterized as follows:

| | | |
|---|---|---|
| viscosity: | 50 | mPas (25° C.) |
| NCO content: | 32.6% | by weight |
| monomer (B1) content: | 49.1% | by weight |
| uretonimine content: | 1.6% | by weight |
| color number: | 0.8 | iodine |

| | |
|---|---|
| L* value: | 99.3 |
| b* value: | 5.1 |

Content and ratio of the higher homologues of diphenylmethane diisocyanate:
(B2) the 3-ring product of polyphenylenepolymethylene polyisocyanate: 30.4% by weight
(B3) the 4-ring product of polyphenylenepolymethylene polyisocyanate: 11.4% by weight
(B4) the 5-ring product of polyphenylenepolymethylene polyisocyanate: 4.2% by weight
   giving a ratio of the constituents (B1):(B2):(B3)=7.2:2.7:1.

The product produced as described above was used in this form as isocyanate component for the following manufacture of 1-component in-situ foam.

1.3 Production of the Light-Colored Dimensionally Stable 1-Component in-situ Foam

268 g of the polyol component from Example 1.1 were introduced into a 1 liter aerosol can. After addition of 361 g of the isocyanate preparation from Example 1.2, the aerosol can was closed by means of a tilt valve using an apparatus suitable for the laboratory manufacture of aerosol cans.

56 g of dimethyl ether, 38 g of a propane/butane mixture composed of 20% by weight of propane and 80% by weight of butane and 94 g of tetrafluoromethane (R134a) were subsequently introduced through the valve and the contents were homogenized by shaking.

Artificial aging was achieved by warm storage of the aerosol can produced in this way at 50° C. so that the aerosol can produced could be tested after storage for 24 hours and cooling to room temperature.

For this purpose, a piece of absorbent paper laid on a flat substrate was moistened and the contents of the aerosol can were discharged as a foaming mixture by actuating the tilt valve with screwed-on foam tube.

The foam was discharged in strips, with wetting of the foam surface with water occurring between the foam strips discharged in layer form.

The cured foam was tested to determine its properties (see Table 1.4).

1.4 Properties of the 1-Component in-situ Foams

| | Foam as described | Comparison with |
|---|---|---|
| | in Example 1 | commercial foam |
| Color | white | yellow/brown |
| Tensile strength | 24.1 N/cm² | 8 N/cm² |
| Elongation at break | 21% | 20% |
| Compressive stress | 5 N/cm² | 5 N/cm² |
| (10% deformation) | | |
| Dimensional stability* | no shrinkage | −4.2% |

Dimensional stability*
The dimensional stability was measured on test specimens made up of two chipboard plates plus spacer rods and foam introduced and cured in between. After curing of the foam and removal of the spacer rods, the percentage change in the spacing between the plates was determined as a measure of the dimensional stability.

### EXAMPLE 2

### Two-Component Rigid PUR Foam

### 2.1 Preparation of the Polyol Component

A polyol mixture was prepared from 377 g of Lupranol 3424 (polyether polyol based on sucrose, pentaerythritol, diethylene glycol and propylene oxide and having an OH number of 403 mg KOH/g), 230 g of Lupranol 3423 (polyether polyol based on sucrose, glycerol and propylene oxide and having an OH number of 490 mg KOH/g), 20 g of glycerol, 300 g of Lupranol 1100 (polyether polyol based on propylene glycol and propylene oxide and having an OH number of 104 mg KOH/g), 54 g of Lupranol VP9319 (polyether polyol based on trimethylolpropane and propylene oxide and having an OH number of 160 mg KOH/g), 10 g of stabilizer Tegostab 88443, 5 g of stabilizer Niax Silicone SR 393 and 4.5 g of water. 34 g of a catalyst mixture (23.3% of N,N-dimethylcyclohexylamine, 18.7% of 1-methylimidazole, 28% of tetramethylhexane-diamine and 30% of Lupranol 1200 [polyether polyol based on propylene glycol and propylene oxide and having an OH number of 248 mg KOH/g]) and also 50 g of an aqueous glycerol/glycol mixture (comprising 9% of glycerol and 31% of dipropylene glycol) were added to this mixture and the polyol component was produced therefrom.

### 2.2 Isocyanate Component

The isocyanate component as described in Example 1.2 was used.

### 2.3 Processing of the Components to Produce Rigid PUR Foam

The components as described in Example 2.1 and 2.2 were mixed in a mixing ratio of polyol component:isocyanate component=100:136, and a white rigid foam was obtained after foaming and curing. The foam (free-foam) had the following properties:

| | | |
|---|---|---|
| cream time: | 15 | sec |
| fiber time: | 48 | sec |
| rise time: | 85 | sec |
| foam density: | 37.2 | kg/m³ |
| compressive strength: | 28.1 | N/cm² |

### 2.4 Comparison of the Dimensional Stability

Using a polyol component having a composition analogous to Example 2.1, rigid foam sandwich boards are, for example, produced firstly using an isocyanate component analogous to the composition described in Example 1.2 and secondly using commercially available polyphenylenepolymethylene polyisocyanate (trade name: Lupranat® M20) and their shrinkage after curing is measured.

| | | Foam using Lupranat ® |
|---|---|---|
| | Foam as described | M20 (polyol component as |
| | in Example 2.3 | described in Example 2.1) |
| Dimensional stability | ±0 (no shrinkage) | −0.2%* |
| (width) | | |
| Dimensional stability | ±0 (no shrinkage) | −0.6%* |
| (length) | | |

*figures from technical information on Elastopor H 1101/1/0

1. A polyphenylenepolymethylene polyisocyanate (B) comprising
(B1) the 2-ring product of polyphenylenepolymethylene polyisocyanate
(B2) the 3-ring product of polyphenylenepolymethylene polyisocyanate
(B3) the 4-ring product of polyphenylenepolymethylene polyisocyanate
(B4) the 5-ring product of polyphenylenepolymethylene polyisocyanate,
wherein the constituents (B2), (B3) and (B4) are, at a content of (B1) of from 2 to 55% by weight, based on the weight of (B), present in a weight ratio of (B2):(B3):(B4) of 8±4:3.5±1.8:1.2±0.9 and the component (B) comprises at least 85% by weight, based on the weight of the component (B), of the constituents (B1), (B2), (B3) and (B4) and not more than 15% by weight, based on the weight of the component (B), of polyphenylenepolymethylene polyisocyanate having at least 6 rings and other compounds comprising isocyanate groups, and the other compounds comprising isocyanate groups comprise uretonimines.

2. The polyphenylenepolymethylene polyisocyanate according to claim 3, wherein the other compounds comprising isocyanate groups comprise uretonimines in an amount of not more than 11% by weight, based on the weight of the polyphenylenepolymethylene polyisocyanate (B).

3. The polyphenylenepolymethylene polyisocyanate according to claim 3, wherein the other compounds comprising isocyanate groups comprise uretonimines in an amount of not more than 6% by weight, based on the weight of the polyphenylenepolymethylene polyisocyanate (B).

4. The polyphenylenepolymethylene polyisocyanate according to claim 3, wherein the other compounds comprising isocyanate groups comprise uretonimines in an amount of not more than 3% by weight, based on the weight of the polyphenylenepolymethylene polyisocyanate (B).

5. The polyphenylenepolymethylene polyisocyanate according to claim 1, wherein the polyphenylenepolymethylene polyisocyanate has a content of free NCO end groups of from 31.0 to 33.3% by weight.

6. A process for producing polyurethane foams by reacting (A) compounds having at least two hydrogen atoms which are reactive toward isocyanate groups with (B) polyisocyanates, wherein the polyphenylenepolymethylene polyisocyanate according to claim 1 is used as polyisocyanate (B).

7. The process according to claim 9, wherein the reaction is carried out in the presence of blowing agents.

8. The process according to claim 9, wherein the reaction is carried out in the presence of catalysts.

9. A process for producing 1-component polyurethane foams by reacting (A) compounds having at least two hydrogen atoms which are reactive toward isocyanate groups with (B) polyisocyanates by mixing the components (A) and (B) in the presence of blowing agents in a pressure container, wherein polyphenylenepolymethylene polyisocyanate according to claim 1 is used as polyisocyanate (B).

10. The process according to claim 12, wherein the component (B) is used in an at least three-fold stoichiometric excess in the reaction of the components (A) and (B).

11. A process for producing rigid polyurethane foams by reacting (A) compounds having at least two hydrogen atoms which are reactive toward isocyanate groups with (B) polyisocyanates in the presence of blowing agents, wherein polyphenylenepolymethylene polyisocyanate according to claim 1 is used as polyisocyanate (B).

12. The process according to claim 14, wherein the reaction of the components (A) and (B) is carried out at an index of from 100 to 220.

13. The process according to claim 14, wherein the reaction is carried out by the two-component process.

14. A process for producing rigid polyurethane-polyisocyanurate foams by reacting (A) compounds having at least two hydrogen atoms which are reactive toward isocyanate groups with (B) polyisocyanates in the presence of blowing agents and trimerization catalysts, wherein polyphenylenepolymethylene polyisocyanate according to claim 1 is used as polyisocyanate (B).

15. The process according to claim 17, wherein the reaction of the components (A) and (B) is carried out at an index of from 160 to 450.

16. A process for preparing polyphenylenepolymethylene polyisocyanate according to claim 1 by reacting polyphenylenepolymethylenepolyamine with phosgene, wherein a temperature of 220° C. is not exceeded during the preparation and work-up of the polyphenylenepolymethylene polyisocyanate.

17. The use of polyphenylenepolymethylene polyisocyanate according to claim 1 for producing polyurethane foams.
